(19) United States
(12) Patent Application Publication
(10) Pub. No.: US 2022/0338842 A1
(43) Pub. Date: Oct. 27, 2022

(54) METHODS AND APPARATUSES FOR PROVIDING INDICATIONS OF MISSING LANDMARKS IN ULTRASOUND IMAGES

(71) Applicant: BFLY Operations, Inc., Guilford, CT (US)

(72) Inventors: Cristina Shin, San Francisco, CA (US); Audrey Howell, Guilford, CT (US); Igor Lovchinsky, New York, NY (US); Israel Malkin, New York, NY (US); Ardavan Saeedi, Jersey City, NJ (US)

(73) Assignee: BFLY Operations, Inc., Guilford, CT (US)

(21) Appl. No.: 17/586,508

(22) Filed: Jan. 27, 2022

Related U.S. Application Data
(60) Provisional application No. 63/143,699, filed on Jan. 29, 2021.

(51) Int. Cl. A61B 8/08; G06T 7/00; A61B 8/00

(52) U.S. Cl. CPC A61B 8/5215; A61B 8/5207; A61B 8/5269; G06T 7/0012; A61B 8/469

(57) ABSTRACT

Methods and apparatuses for providing indications of missing landmarks in ultrasound images are described. Some embodiments are directed to apparatuses comprising a processing device configured to obtain data representing an ultrasound image, and determine whether the ultrasound image is clinically usable, wherein the determining comprises determining whether the ultrasound image lacks one or more landmarks. Determining whether the ultrasound image is clinically usable may further comprise determining a quality value representative of a quality of the ultrasound image and comparing the quality value to a threshold quality value. In some embodiments, landmarks comprise one or more anatomical features, such as a rib, a pleural line and an A line, a liver, and a kidney.

### CROSS-REFERENCE TO RELATED APPLICATIONS

This application claims the benefit under 35 U.S.C. § 119(e) of U.S. Provisional Patent Application Ser. No. 63/143,699, filed Jan. 29, 2021, under Attorney Docket No. B1348.70193US00 and entitled “METHODS AND APPARATUSES FOR PROVIDING INDICATIONS OF MISSING LANDMARKS IN ULTRASOUND IMAGES,” which is hereby incorporated by reference herein in its entirety.

### FIELD

Generally, the aspects of the technology described herein relate to ultrasound images. Some aspects relate to providing indications of missing landmarks in ultrasound images.

### BACKGROUND

Ultrasound probes may be used to perform diagnostic imaging and/or treatment, using sound waves with frequencies that are higher than those audible to humans. Ultrasound imaging may be used to see internal soft tissue body structures. When pulses of ultrasound are transmitted into tissue, sound waves of different amplitudes may be reflected back towards the probe at different tissue interfaces. These reflected sound waves may then be recorded and displayed as an image to the operator. The strength (amplitude) of the sound signal and the time it takes for the wave to travel through the body may provide information used to produce the ultrasound image. Many different types of images can be formed using ultrasound devices. For example, images can be generated that show two-dimensional cross-sections of tissue, blood flow, motion of tissue over time, the location of blood, the presence of specific molecules, the stiffness of tissue, or the anatomy of a three-dimensional region.

### SUMMARY

According to one aspect of the application, an apparatus comprises a processing device configured to: generate an ultrasound image for display; determine a quality of the ultrasound image; determine whether the quality of the ultrasound image is below a threshold quality; if the processing device determines that the quality of the ultrasound image is not below the threshold quality, provide an indication of the quality of the ultrasound image; and if the processing device determines that the quality of the ultrasound image is below the threshold quality: determine one or more missing landmarks in the ultrasound image; and provide an indication of the quality of the ultrasound image and an indication of the one or more missing landmarks in the ultrasound image.

In some embodiments, the one or more missing landmarks comprise one or more anatomical features that, if present in the ultrasound image, would make the ultrasound image clinically usable.

In some embodiments, the ultrasound image comprises an ultrasound image of lungs, and the one or more missing landmarks comprise one or more of ribs, a pleural line, and A lines.

In some embodiments, the ultrasound image comprises an ultrasound image of Morison's pouch, and the one or more missing landmarks comprise on or more of a liver and a kidney.

In some embodiments, the processing device is configured, when providing the indication of the one or more missing landmarks in the ultrasound image, to display a number of the missing landmarks.

In some embodiments, the processing device is configured, when providing the indication of the one or more missing landmarks in the ultrasound image, to display a number of total landmarks that, if present in the ultrasound image, would make the ultrasound image clinically usable as well as a number of landmarks present in the ultrasound image.

In some embodiments, the processing device is configured, when providing the indication of the one or more missing landmarks in the ultrasound image, to display a reference ultrasound image that includes all landmarks that, if present in the ultrasound image, would make the ultrasound image clinically usable.

In some embodiments, the processing device is further configured to highlight the missing landmarks in the reference ultrasound image.

In some embodiments, the processing device is configured, when providing the indication of the one or more missing landmarks in the ultrasound image, to display a schematic representation of an ultrasound image that includes schematic representations of all landmarks that, if present in the ultrasound image, would make the ultrasound image clinically usable.

In some embodiments, the processing device is further configured to highlight schematic representations of the missing landmarks in the schematic representation of the ultrasound image.

In some embodiments, the processing device is configured, when providing the indication of the one or more missing landmarks in the ultrasound image, to display a checklist indicating the one or more missing landmarks and landmarks present in the ultrasound image.

In some embodiments, the processing device is configured, when determining the quality of the ultrasound image, to determine the quality based on a predicted proportion of experts who would consider the ultrasound image to be clinically usable.

According to one aspect of the application, an apparatus comprises a processing device configured to: obtain data representing an ultrasound image; and determine whether the ultrasound image is clinically usable, wherein the determining comprises determining whether the ultrasound image lacks one or more landmarks.

In some embodiments, obtaining the data comprises: receiving the data from an ultrasound device electrically connected to the processing device.

In some embodiments, determining whether the ultrasound image is clinically usable further comprises determining a quality value representative of a quality of the ultrasound image and comparing the quality value to a threshold quality value.

In some embodiments, the one or more landmarks comprise one or more anatomical features.

In some embodiments, the ultrasound image comprises an ultrasound image of at least one lung, and the one or more anatomical features comprise at least one selected from the group consisting of a rib, a pleural line and an A line.

In some embodiments, the ultrasound image comprises an ultrasound image of a Morison's pouch, and the one or more anatomical features comprise at least one selected from the group consisting of a liver and a kidney.

In some embodiments, the processing device is further configured to: cause a display to display the ultrasound image including information indicative of the one or more lacking landmarks.

In some embodiments, the information indicative of the one or more lacking landmarks comprises text indicative of the one or more lacking landmarks.

In some embodiments, the information indicative of the one or more lacking landmarks comprises a reference depiction of the one or more lacking landmarks.

In some embodiments, the processing device is further configured to: determine a quality value representative of a quality of the ultrasound image, and cause a display to display the ultrasound image including information indicative of the quality of the ultrasound image based on quality value.

In some embodiments, determining the quality value comprises applying, to a statistical model, data indicative of a predicted proportion of experts who would consider the ultrasound image to be clinically usable.

According to one aspect of the application, a method comprises obtaining data representing an ultrasound image; and determining whether the ultrasound image is clinically usable, wherein the determining comprises determining whether the ultrasound image lacks one or more landmarks.

In some embodiments, obtaining the data comprises: receiving the data from an ultrasound device electrically connected to the processing device.

In some embodiments, determining whether the ultrasound image is clinically usable further comprises determining a quality value representative of a quality of the ultrasound image and comparing the quality value to a threshold quality value.

In some embodiments, the one or more landmarks comprise one or more anatomical features.

In some embodiments, the ultrasound image comprises an ultrasound image of at least one lung, and the one or more anatomical features comprise at least one selected from the group consisting of a rib, a pleural line and an A line.

In some embodiments, the ultrasound image comprises an ultrasound image of a Morison's pouch, and the one or more anatomical features comprise at least one selected from the group consisting of a liver and a kidney.

In some embodiments, the method further comprises causing a display to display the ultrasound image including information indicative of the one or more lacking landmarks.

In some embodiments, the information indicative of the one or more lacking landmarks comprises text indicative of the one or more lacking landmarks.

In some embodiments, the information indicative of the one or more lacking landmarks comprises a reference depiction of the one or more lacking landmarks.

In some embodiments, the method further comprises determining a quality value representative of a quality of the ultrasound image, and causing a display to display the ultrasound image including information indicative of the quality of the ultrasound image based on quality value.

In some embodiments, determining the quality value comprises applying, to a statistical model, data indicative of a predicted proportion of experts who would consider the ultrasound image to be clinically usable.

### BRIEF DESCRIPTION OF THE DRAWINGS

Various aspects and embodiments will be described with reference to the following exemplary and non-limiting figures. It should be appreciated that the figures are not necessarily drawn to scale. Items appearing in multiple figures are indicated by the same or a similar reference number in all the figures in which they appear.

FIG. 1 is a flow diagram illustrating an example process 100 for providing an indication of missing landmarks in an ultrasound image, in accordance with certain embodiments described herein.

FIGS. 2-9 illustrate representative graphical user interfaces (GUIs), in accordance with certain embodiments described herein.

FIG. 10 illustrates a schematic block diagram of an example ultrasound system), in accordance with certain embodiments described herein.

### DETAILED DESCRIPTION

Aspects of the present application relate to techniques for determining whether an ultrasound image provides sufficient information to make the image clinically usable (e.g., usable for purposes of medical diagnostics). Some embodiments relate to statistical models for determining the quality of an ultrasound image. For example, the quality of the ultrasound image may be based on a prediction of what proportion of experts (e.g., experts in the field of medicine, experts in a particular field of medicine, experts in ultrasound imaging, etc.) would consider the ultrasound image clinically usable. A processing device that displays an ultrasound image may use such a statistical model to determine the quality of the ultrasound image and display an indication of the quality on a graphical user interface (GUI). The inventors have recognized that, when the quality of the ultrasound image is below a certain threshold quality, instead of only providing an indication of the quality of the ultrasound image, it may be helpful for the processing device to also indicate why the quality of the ultrasound image may be below the threshold quality. According to some aspects of the present application, the indication may be related to landmarks present in, or absent from, the ultrasound image.

In some embodiments, landmarks may include any type of anatomical feature, such as an anatomical region or structure, that when present in an ultrasound image, may be viewed as an indication that the ultrasound image is clinically usable. For example, an ultrasound image of the lungs may be deemed clinically usable for certain purposes when the ultrasound image includes two ribs, the pleural line, and A lines. As another example, an ultrasound image of Morison's pouch may be deemed clinically usable when the ultrasound image includes the liver and kidney. The presence or absence of such landmarks may be correlated with the quality level of the ultrasound image determined by the statistical model described above. The inventors have recognized that providing an indication of missing landmarks for an ultrasound image determined by the processing device to have a quality below a threshold quality may help the user obtain a higher quality ultrasound image. The inventors have further developed various GUIs for displaying indications of missing landmarks.

Various aspects of the present disclosure may be used alone, in combination, or in a variety of arrangements, and is therefore not limited in its application to the details and arrangement of components set forth in the foregoing description or illustrated in the drawings. For example, aspects described in one embodiment may be combined in any manner with aspects described in other embodiments.

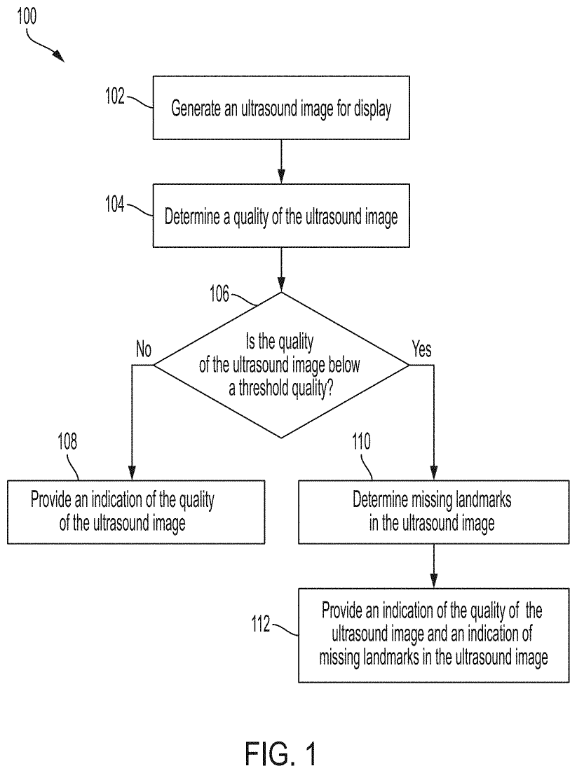

FIG. 1 is a flow diagram illustrating an example process 100 for providing an indication of missing landmarks in an ultrasound image, in accordance with certain embodiments described herein. The process 100 may be performed by a processing device, such as a mobile phone, tablet, or laptop. The processing device may be in operative communication with an ultrasound device. The ultrasound device and the processing device may communicate over a wired communication link (e.g., over Ethernet, a Universal Serial Bus (USB) cable or a Lightning cable) or over a wireless communication link (e.g., over a BLUETOOTH, WiFi, or ZIGBEE wireless communication link). In some embodiments, the processing device may be part of the ultrasound device. The process 100 begins at act 102.

In act 102, the processing device generates an ultrasound image for display. In some embodiments, the processing device may generate the ultrasound image for display on a display screen of the processing device. In some embodiments, the ultrasound device may have collected raw ultrasound data and transmitted the raw ultrasound data and/or scan lines generated based on the raw ultrasound data to the processing device, and the processing device may have generated the ultrasound image from the ultrasound data and then may generate the ultrasound image for display. In some embodiments, the ultrasound device may have generated the ultrasound image based on the ultrasound data and transmitted the ultrasound image to the processing device, and the processing device may then generate the ultrasound image for display. In some embodiments, the processing device may generate the ultrasound image for display in real-time as ultrasound imaging is being performed. In some embodiments, the ultrasound image may have been previously stored to memory, and the processing device may have retrieved the ultrasound image from the memory and may then generate the ultrasound image for display. For example, the processing device may have retrieved the ultrasound image from a temporary storage buffer on the processing device, from permanent memory on the processing device, or from an external device (e.g., a server).

In act 104, the processing device determines a quality of the ultrasound image (e.g., the ultrasound image generated for display in act 102). In some embodiments, the quality of the ultrasound image may be based on a prediction of what proportion of experts (e.g., experts in the field of medicine, experts in a particular field of medicine, experts in ultrasound imaging, etc.) would consider the ultrasound image clinically usable. In some embodiments, to determine the quality of the ultrasound image, the processing device may use a statistical model trained to output such a prediction based on an inputted ultrasound. Further description of determining and the quality of an ultrasound image may be found in U.S. Patent Application Publication No. 2020/0372657 titled “METHODS AND APPARATUSES FOR ANALYZING IMAGING DATA,” filed on May 21, 2020 (and assigned to the assignee of the instant application), which is incorporated by reference herein in its entirety.

In act 106, the processing device determines if the quality of the ultrasound image (determined in act 104) is less than a threshold quality. As described above, in some embodiments the quality of the ultrasound image may be based on the predicted proportion of experts who would consider the ultrasound image to be clinically usable. Thus, the threshold quality may be a threshold proportion of experts who would consider the ultrasound image to be clinically usable. As one example, the threshold quality (and thus the threshold proportion) may be one-third. As another example, the threshold quality (and thus the threshold proportion) may be two-thirds. However, other threshold qualities may be used. If, at act 106, the processing device determines that the quality of the ultrasound image is not less than the threshold quality, then the process 100 proceeds to act 108. If, at act 106, the processing device determines that the quality of the ultrasound image is less than the threshold quality, then the process 100 proceeds to act 110.

In act 108, the processing device provides an indication of the quality of the ultrasound image. As described above, in some embodiments the quality of the ultrasound image may be based on the predicted proportion of experts who would consider the ultrasound image to be clinically usable. Thus, in some embodiments, the processing device may provide an indication of the predicted proportion of experts who would consider the ultrasound image to be clinically usable. In some embodiments, the processing device may display the proportion as a number. In some embodiments, the processing device may display the proportion graphically. In some embodiments, the processing device may provide an indication of one of multiple quality levels. As one non-limiting example, if the predicted proportion is equal to or between 0-32%, then the processing device may provide an indication of low quality. If the predicted proportion is equal to or between 33-65%, then the processing device may provide an indication of medium quality. If the predicted proportion equal to or between 66-100%, then the processing device may provide an indication of high quality. According to some embodiments, if the predicted proportion is equal to or between 0-50%, then the processing device may provide an indication of insufficient quality, and if the predicted proportion is equal to or between 51-100%, then the processing device may provide an indication of sufficient quality. In some embodiments, the processing device may display the quality level textually (e.g., by displaying the text “Low quality,” “Medium quality,” or “High quality”). In some embodiments the processing device may display the quality level graphically. It should be appreciated that other ranges may be used for these three levels of quality, and while the above example uses three levels, other numbers of levels may be used as well. Examples of graphical user interfaces (GUIs) that the processing device may display at act 108 include the GUIs 200, 400, 700, 800, and 900 described below.

As described above, if the processing device determines at act 106 that the quality of the ultrasound image is less than the threshold quality, then the process 100 proceeds to act 110. In act 110, the processing device determines one or more missing landmarks in the ultrasound image. Landmarks may be any type of anatomical feature, such as an anatomical region or structure, that when present in an ultrasound image, may be viewed as an indication that the ultrasound image is clinically usable. For example, an ultrasound image of the lungs may be deemed clinically usable for certain purposes when the ultrasound image includes two ribs, the pleural line, and A lines. As another example, an ultrasound image of Morison's pouch may be deemed clinically usable when the ultrasound image includes the liver and at least one kidney. Similarly, an ultrasound image of a right upper quadrant (RUQ) may be deemed clinically usable when the ultrasound image includes the liver and at least one kidney. As yet another example, an ultrasound image showing a cardiac view (e.g., an apical 4 chamber view) may be deemed clinically usable when the ultrasound image includes the right ventricle (RV), the right atrium (RA), the left ventricle (LV), the left atrium (LA), the mitral valve (MV) and a tricuspid valve (TV). As yet another example, an ultrasound image showing another cardiac view (e.g., a parasternal long axis view (PLAX)) may be deemed clinically usable when the ultrasound image includes the left atrium, the left ventricle, the aortic valve, the mitral valve, the right ventricular outflow tract (RVOT), the descending thoracic aorta (DTA) and the aortic root (AR). As yet another example, an ultrasound image showing yet another cardiac view (e.g., a parasternal short axis view (PSAX)) may be deemed clinically usable when the ultrasound image includes the right ventricle, the left ventricle, a more papillary muscle, the mitral valve and the aortic valve. As yet another example, an ultrasound image showing yet another cardiac view (e.g., a subxiphoid view) may be deemed clinically usable when the ultrasound image includes the right ventricle, the left ventricle, the right atrium, the left atrium and the liver. As yet another example, an ultrasound image showing a splenorenal recess may be deemed clinically usable when the ultrasound image includes the spleen and at least one kidney. Similarly, an ultrasound image of a left upper quadrant (LUQ) may be deemed clinically usable when the ultrasound image includes the spleen and at least one kidney. As yet another example, an ultrasound image showing a pelvic view may be deemed clinically usable when the ultrasound image includes the bladder. Similarly, an ultrasound image of a bladder may be deemed clinically usable when the ultrasound image includes the entire bladder or at least a fraction of the bladder. As yet another example, an ultrasound image of the carotid artery may be deemed clinically usable when the ultrasound image includes the common carotid artery and the carotid bulb. As yet another example, an ultrasound image of the femoral artery may be deemed clinically usable when the ultrasound image includes the common femoral artery and the femoral bulb.

In some embodiments, an ultrasound image showing a particular view may be deemed clinically usable even if includes a subset of the anatomic features listed above as associated with that view. For example, an ultrasound image showing a subxiphoid view may be deemed clinically usable when the ultrasound image includes the right ventricle, the left ventricle, the right atrium and the liver, but not the left atrium. Further, the presence of additional anatomical features in a particular view may not affect the clinical usability of that view. Accordingly, some views may include more anatomic features than those listed above in connection with that view, and yet, that view may be deemed clinically usable.

In some embodiments, the processing device may use a statistical model to determine the presence or absence of landmarks. In such embodiments, the processing device may use a statistical model trained to determine the locations of particular landmarks as depicted in ultrasound images. The statistical model may be stored on the processing device or stored on another electronic device (e.g., a server) and accessed by the processing device. In some embodiments, the statistical model may be trained on multiple pairs of input and output training data sets as a segmentation model. Each set of input training data may be an ultrasound image depicting one or more landmarks. Each set of output training data may include multiple segmentation masks for each of the landmarks. Each segmented mask may include an array of values equal in size to the input training data ultrasound image, and pixels corresponding to locations within one of the landmarks in the ultrasound image are manually set to 1 and other pixels are set to 0. Based on this training data, the statistical model may learn to output, based on an inputted ultrasound image, one or more segmentation masks, where each pixel in a given mask has a value representing the probability that the pixel corresponds to a location within a landmark in the ultrasound image (values closer to 1) or outside the landmark (values closer to 0). The processing device may select all pixels in a given segmentation mask that have a value greater than a threshold value (e.g., 0.5) as being within a landmark. The processing device may do this for all the segmentation masks in order to determine the locations of multiple landmarks in the ultrasound image.

In some embodiments, the processing device may determine the presence or absence of landmarks based on segmentation masks. In some embodiments, the processing device may analyze a segmentation mask to determine if the corresponding landmark is present in the ultrasound image using various heuristics. For example, using heuristics may include determining whether the number of pixels determined to be within the landmark in the segmentation mask (“segmented pixels”) is greater than a threshold number and/or analyzing various other relationships between the segmented pixels, such as how continuous they are (e.g., using connected components analysis).

In some embodiments, the processing device may use the quality of the ultrasound image to determine the presence or absence of landmarks. For example, if the quality of the ultrasound image (e.g., as determined in act 104) is lower than a threshold quality, then the processing device may assume that no landmarks are present in the ultrasound image. The process 100 proceeds to act 112.

In act 112, the processing device provides an indication of the quality of the ultrasound image and an indication of the presence or absence of landmarks in the ultrasound image (i.e., as determined in act 110). Further description of providing an indication of the quality of an ultrasound image may be found with reference to act 108. The inventors have recognized that, when the quality of the ultrasound image is below a certain threshold quality, in addition to (or instead of) providing an indication of the quality of the ultrasound image, it may be helpful for the processing device to also indicate why the quality of the ultrasound image may be below the threshold quality. In some embodiments, the presence or absence of landmarks may be correlated with the quality level of the ultrasound image determined by the statistical model described above. The inventors have recognized that providing an indication of the presence or absence of landmarks for an ultrasound image determined by the processing device to have a quality below a threshold quality may help the user obtain a higher quality ultrasound image. Examples of graphical user interfaces (GUIs) that the processing device may display at act 112 include the GUIs 300, 400, 500, and 600.

In some embodiments (of which the GUI 300 is an example), the processing device may provide an indication of missing landmarks by displaying the number of landmarks missing in the ultrasound image. In some embodiments (of which the GUI 300 is an example), the processing device may provide an indication of missing landmarks by displaying the number of total landmarks that, if present, may make the ultrasound image clinically usable, as well as the number of landmarks present in the ultrasound image. In some embodiments (of which the GUI 400 is an example), the processing device may provide an indication of missing landmarks by providing a reference ultrasound image that includes all the landmarks that, if present, may make the ultrasound image clinically usable. In some embodiments, the reference ultrasound image may highlight those landmarks missing in the collected ultrasound image. For example, highlighting the landmarks may include displaying, with a specific format (e.g., a specific color) shapes on the landmarks in the reference ultrasound image and/or displaying, with a specific format (e.g., a specific color) labels adjacent to the landmarks in the reference ultrasound image. In some embodiments (of which the GUI 500 is an example), the processing device may provide an indication of missing landmarks by displaying a schematic representation of an ultrasound image. The schematic representation of the ultrasound image may include schematic representations of all the landmarks that, if present, may make the ultrasound image clinically usable, such that the arrangement of the schematic representations of landmarks relative to the schematic representation of the ultrasound image and relative to each other may match, at least approximately, the arrangement of the corresponding landmarks relative to an actual ultrasound image and relative to each other. In some embodiments, the schematic representation of the clinically usable ultrasound image may highlight the schematic representations of landmarks corresponding to those landmarks missing in the collected ultrasound image. For example, highlighting the landmarks may include displaying the schematic representations of the landmarks with a specific format (e.g., a specific color). In some embodiments (of which the GUI 600 is an example), the processing device may provide a checklist indicating landmarks present and missing in the ultrasound image.

In some embodiments, act 102 may be absent. In other words, the processing device may not generate the ultrasound image for display, but may still perform the determinations for the ultrasound image in acts 104, 106, and 110, and provide the indications in acts 108 or act 112. In some embodiments, at act 104, the processing device may retrieve the quality of the ultrasound image that has been determined by another device rather than performing the determination itself. In some embodiments, at act 106, the processing device may determine whether the quality of the ultrasound image is less than or equal to a threshold quality. In some embodiments, at act 106, the processing device may determine whether the quality of the ultrasound image is greater than a threshold quality, proceed to act 108 if it is, and proceed to act 110 if it is not. In some embodiments, at act 106, the processing device may determine whether the quality of the ultrasound image is greater than or equal to a threshold quality, proceed to act 108 if it is, and proceed to act 110 if it is not. In some embodiments, act 108 may be absent. In other words, the processing device may not provide an indication of the quality of the ultrasound image when the ultrasound image is not below the threshold quality. In some embodiments, at act 110, the processing device may retrieve information regarding missing landmarks from another device rather than performing the determination itself. In some embodiments, at act 112, the processing device may provide the indication of the missing landmarks but not the indication of the quality of the ultrasound image.

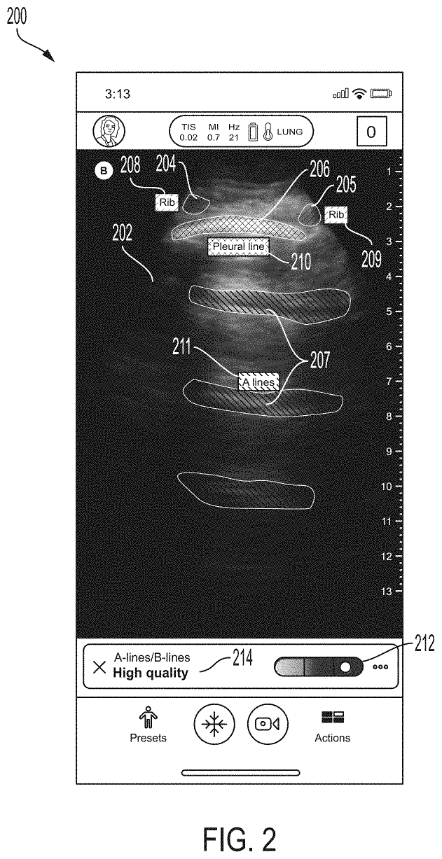

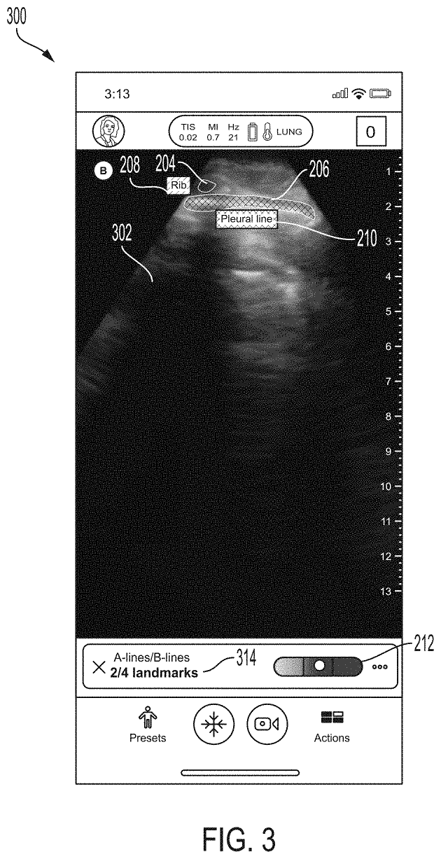

FIGS. 2-9 illustrate example graphical user interfaces (GUIs) that may be displayed by a processing device in an ultrasound system, in accordance with certain embodiments described herein. The processing device may be, for example, a mobile phone, tablet, or laptop. The processing device may be in operative communication with an ultrasound device, and the ultrasound device and the processing device may communicate over a wired communication link (e.g., over Ethernet, a Universal Serial Bus (USB) cable or a Lightning cable) or over a wireless communication link (e.g., over a BLUETOOTH, WiFi, or ZIGBEE wireless communication link). In some embodiments, the ultrasound device itself may display the GUIs. It should be appreciated that the forms of the GUIs illustrated in the figures are non-limiting, and other GUIs displaying the same information with different forms may also be used. The GUIs illustrated in FIGS. 2-9 may be displayed in conjunction with the process 100. In particular, the GUIs illustrated in FIGS. 2, 4, 7, 8, and 9 may be displayed in conjunction with act 108 and the GUIs illustrated in FIGS. 3, 4, 5, and 6 may be displayed in conjunction with act 112.

FIG. 2 illustrates a GUI 200. The GUI 200 includes an ultrasound image 202, segmented landmarks 204-207, labels 208-211, a quality indicator 212, and a quality indicator 214.

In some embodiments, the ultrasound image 202 may be displayed in real-time as ultrasound imaging is being performed. For example, the ultrasound device may have collected raw ultrasound data and transmitted the raw ultrasound data and/or scan lines generated based on the raw ultrasound data to the processing device, and the processing device may have generated the ultrasound image 202 from the ultrasound data and displayed the ultrasound image 202. As another example, the ultrasound device may have generated the ultrasound image 202 based on the ultrasound data and transmitted the ultrasound image 202 to the processing device, and the processing device may have displayed the ultrasound image 202. In some embodiments, the ultrasound image 202 may have been previously stored to memory, and the processing device may have retrieved the ultrasound image 202 from the memory. For example, the processing device may have retrieved the ultrasound image 202 from a temporary storage buffer on the processing device, from permanent memory on the processing device, or from an external device (e.g., a server). In some embodiments, the ultrasound image 202 may be part of a cine that was previously stored to memory, and the processing device may have retrieved the cine from the memory and displayed ultrasound images in the cine one after another.

The quality indicator 212 may graphically indicate a quality of the ultrasound image 202. In some embodiments, the quality of the ultrasound image 202 may be based on a prediction of what proportion of experts (e.g., experts in the field of medicine, experts in a particular field of medicine, experts in ultrasound imaging, etc.) would consider the ultrasound image 202 clinically usable. In some embodiments, to determine the quality of the ultrasound image 202, the processing device may use a statistical model trained to output such a prediction based on an inputted ultrasound image. The quality indicator 212 in FIG. 2 graphically displays this quality using a bar and a circle, where the distance of the circle from one end of the bar relative to the full length of the bar is proportional to the quality. Additionally, vertical lines through the bar may divide the bar into three portions that may indicate three different levels of quality. For example, if the circle is in the leftmost section, then that may indicate that the ultrasound image is of low quality. If the circle is in the middle section, then that may indicate that the ultrasound image is of medium quality. If the circle is in the rightmost section, then that may indicate that the ultrasound image is of high quality. Other numbers of quality levels, and therefore numbers of sections of the bar, may be used instead. Generally, other forms for graphically displaying the quality may be used. Further description of determining and displaying the quality of an ultrasound image may be found in U.S. Patent Application Publication No. 2020/0372657 titled “METHODS AND APPARATUSES FOR ANALYZING IMAGING DATA,” filed on May 21, 2020 (and assigned to the assignee of the instant application).

The quality indicator 214 may indicate a quality of the ultrasound image 202 using text. In some embodiments, the quality indicated by the quality indicator 214 may be based on levels of the predicted proportion of experts who would consider the ultrasound image 202 clinically usable, as described above. As one non-limiting example, if the predicted proportion is equal to or between 0-32%, then the quality indicator 214 may indicate “Low quality.” If the predicted proportion is equal to or between 33-65%, then the quality indicator 214 may indicate “Medium quality.” If the predicted proportion equal to or between 66-100%, then the quality indicator 214 may indicate “High quality.” It should be appreciated that other ranges may be used for these three levels of quality, and while the above example used three levels, other numbers of levels may be used as well.

The segmented landmarks 204-207 are shapes that are superimposed on particular portions of the ultrasound image 202 that correspond to particular landmarks, such as anatomical regions or structures. The ultrasound image 202 is an ultrasound image of a patient's lungs, and the GUI 200 includes a segmented landmark 204 for a rib, a segmented landmark 205 for another rib, a segmented landmark 206 for the pleural line, and segmented landmarks 207 for A lines. The segmented landmarks 204-207 are colored, semi-transparent shapes, with one color for the segmented landmarks 204 and 205 corresponding to the ribs, another color for the segmented landmark 206 corresponding to the pleural line, and another color for the segmented landmarks 207 corresponding to A lines. However, other forms for segmented landmarks are possible, such as non-colored, non-transparent, and outlined forms.

In some embodiments, the processing device may use a statistical model to generate and display the segmented portions 204-207. In particular, the statistical model may be trained to determine the locations for the segmented portions in ultrasound images. For example, a statistical model may be trained to determine the locations of particular landmarks (e.g., ribs, the pleural line, and A lines) as depicted in ultrasound images. The statistical model may be stored on the processing device or stored on another electronic device (e.g., a server) and accessed by the processing device. In some embodiments, the statistical model may be trained on multiple pairs of input and output training data sets as a segmentation model. Each set of input training data may be an ultrasound image depicting one or more landmarks. Each set of output training data may include multiple segmentation masks for each of the landmarks. Each segmented mask may be an array of values equal in size to the input training data ultrasound image, and pixels corresponding to locations within one of the landmarks in the ultrasound image are manually set to 1 and other pixels are set to 0. Based on this training data, the statistical model may learn to output, based on an inputted ultrasound image (e.g., the ultrasound image 202), multiple segmentation masks, where each pixel in a given mask has a value representing the probability that the pixel corresponds to a location within a landmark in the ultrasound image (values closer to 1) or outside the landmark (values closer to 0). The processing device may select all (or some) pixels in a given segmentation mask that have a value greater than a threshold value (e.g., 0.5) as being within a landmark and display a segmented landmark over those pixels in the ultrasound image 202. The processing device may do this for all the segmentation masks in order to display multiple segmented landmarks. In the particular example of ribs in FIG. 2, the statistical model may output a single segmentation mask for all ribs in the ultrasound image 202 and then the processing device may perform image processing (e.g., connected components analysis) to determine how many individual ribs are represented in the segmentation mask and display a different segmented landmark 204 and 205 for each one. While the above description has described a single statistical model configured to output multiple segmentation masks, in some embodiments the processing device may use multiple segmentation models, each configured to output a single segmentation mask. In some embodiments, using a single statistical model may conserve memory and/or power of the processing device. Any of the statistical models described herein may be, for example, a convolutional neural network, a fully connected neural network, a recurrent neural network (e.g., a long short-term memory (LSTM) recurrent neural network), a random forest, a support vector machine, a linear classifier, and/or any other statistical model, and may use deep learning techniques to generate the segmented anatomical portions.

The labels 208-211 label each of the segmented landmarks 204-207. Each of the labels 208-211 is adjacent to the segmented landmark which it labels in the ultrasound image 202. The label 208 displays the text “Rib” and corresponds to the segmented landmark 204, which indicates the location of a rib in the ultrasound image 202. The label 209 displays the text “Rib” and corresponds to the segmented landmark 205, which indicates the location of another rib in the ultrasound image 202. The label 210 displays the text “Pleural Line” and corresponds to the segmented landmark 206, which indicates the location of the pleural line in the ultrasound image 202. The label 210 displays the text “A lines” and corresponds to the segmented landmarks 207, which indicate the locations of A lines in the ultrasound image 202. In the example of FIG. 2, each of the labels 208-211 shares the color of the segmented landmark which it labels. However, in some embodiments, such as those that do not include colored segmented landmarks, the labels 208-211 may not be colored in this way. As described above, in some embodiments the processing device may use a statistical model that outputs multiple segmentation masks in order to generate the multiple segmented landmarks 204-207. In such embodiments, each of the segmentation masks may be associated with one of the labels 208-211, and the processing device may use a particular segmentation mask's label when generating a segmented landmark corresponding to that segmentation mask.

Certain ultrasound images may be clinically usable when they include certain landmarks, such as anatomical regions or structures. For example, an ultrasound image of the lungs, such as the ultrasound image 202, may be clinically usable for certain purposes when it includes two ribs, the pleural line, and A lines. The presence or absence of such landmarks may correlate with the quality level of the ultrasound image determined by the statistical model described above. For example, the presence of two ribs, the pleural line, and A lines in the ultrasound image 202 may correlate with the high quality determined by the statistical model and displayed by the quality indicators 212 and 214.

FIG. 3 illustrates a GUI 300. The GUI 300 includes an ultrasound image 302, the segmented landmarks 204 and 206, the labels 208 and 210, the quality indicator 212, and a landmark indicator 314.

Further description of collection and display of the ultrasound image 302 may be found with reference to the ultrasound image 202. As can be seen, the ultrasound image 302 shows one rib, as indicated by the segmented landmark 204 and the label 208, and the pleural line, as indicated by the segmented landmark 206 and the label 210. However, the ultrasound image 302 lacks a second rib and A lines. As described above, an ultrasound image of the lungs may be clinically usable for certain purposes if it includes two ribs, the pleural line, and A lines. The lack of a rib and A lines in the ultrasound image 302 may correlate with the medium quality indicated by the quality indicator 212.

The inventors have recognized that instead of displaying “Low quality” or “Medium quality” with text (in a similar manner as the quality indicator 214 displays “High quality” in FIG. 2), it may be more helpful for the GUI 300 to indicate why the ultrasound image 302 displayed in the GUI 300 may be low or medium quality. Thus, the landmark indicator 314 indicates that the ultrasound image 302 includes only two of the four landmarks suggested for a clinically usable ultrasound image of the lungs. In other words, the landmark indicator 314 indicates the number of landmarks missing in the ultrasound image, the number of landmarks present in the ultrasound image, and the number of total landmarks that, if present, may make the ultrasound image clinically usable. A user may use the landmark indicator 314 to adjust the ultrasound imaging (e.g., modify the position or orientation of the ultrasound device) to capture a new ultrasound image that includes all four landmarks (e.g., an ultrasound image like the ultrasound image 202). Generally, the processing device may display the GUI 300, which includes the landmark indicator 314, when the quality determined for the displayed ultrasound is below a threshold quality (e.g., in some embodiments, when the ultrasound image has a quality corresponding to “Low quality”, or in some embodiments, when the ultrasound image has a quality corresponding to “Low quality” or “Medium quality”). The processing device may display the GUI 200, which does not include the landmark indicator 314, when the quality determined for the displayed ultrasound is not below threshold quality (e.g., in some embodiments, when the ultrasound image has a quality corresponding to “High quality”, or in some embodiments, when the ultrasound image has a quality corresponding to “High quality” or “Medium quality”).

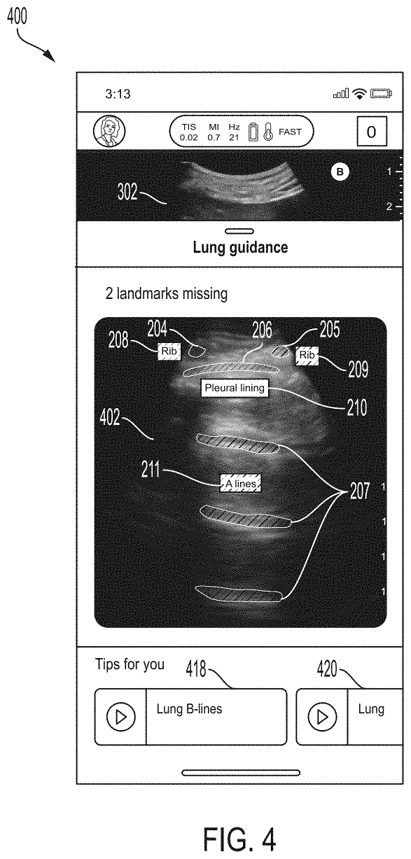

FIG. 4 illustrates a GUI 400. In some embodiments, the processing device may display the GUI 400 upon a selection of the user from the GUI 300. In some embodiments, the selection may include the user selecting (e.g., by touching on a touch-sensitive screen) the region adjacent to the quality indicators 212 and 314. However, in other embodiments, a user may cause the processing device to display the GUI 400 by selecting another portion of the GUI 300, or an option not illustrated in the embodiment of FIG. 3.

The GUI 400 is the same as the GUI 300, except that a reference ultrasound image 402 is displayed on top of the ultrasound image 302. The reference ultrasound image 402 may be a previously-collected ultrasound image, may have been collected by another user using another ultrasound device, and/or may be stored on the processing device or an external device (e.g., a server) accessed by the processing device. The reference ultrasound image 402 may serve as a reference for an ultrasound image of the patient's lungs that includes all the landmarks that may make such an ultrasound image clinically usable for certain purposes. The segmented landmarks 204-207 and the labels 208-211 are superimposed on the ultrasound image 402. In some embodiments, the segmented landmarks 204-207 and the labels 208-211 may have been generated by a different processing device and embedded in the ultrasound image 302 prior to being stored.

In some embodiments, the segmented landmark 205 and the label 209, corresponding to one of the ribs, and the segmented landmarks 207 and the label 211, corresponding to A lines, are highlighted. For example, in FIG. 4, the processing device displays the segmented landmarks 205 and 207 in a different color than the segmented landmarks 204 and 206, and the processing device displays the labels 209 and 211 in a different color than the labels 208 and 210. Highlighting the segmented landmarks and labels corresponding to the rib and A lines may indicate that these landmarks are missing in the ultrasound 302. Generally, the processing device may highlight in the reference ultrasound image those segmented landmarks and labels corresponding to landmarks that are missing in the collected ultrasound image. Referring to the reference ultrasound image 402 and its highlighted portions may help the user collect a clinically usable ultrasound image.

The GUI 400 further includes an indicator 416 of how many landmarks are missing in the ultrasound image 302 as well as options 418 and 420. When a user selects one of the options 418 or 420, the processing device may play a video showing how to collect a clinically usable ultrasound image of the lungs.

In some embodiments, the processing device may display the reference ultrasound image 402 without the user selecting an option. For example, the processing device may display the GUI 400 instead of the GUI 300 without the user selecting an option. As another option, the processing device may display the reference ultrasound image 402 as a thumbnail in the GUI 300.

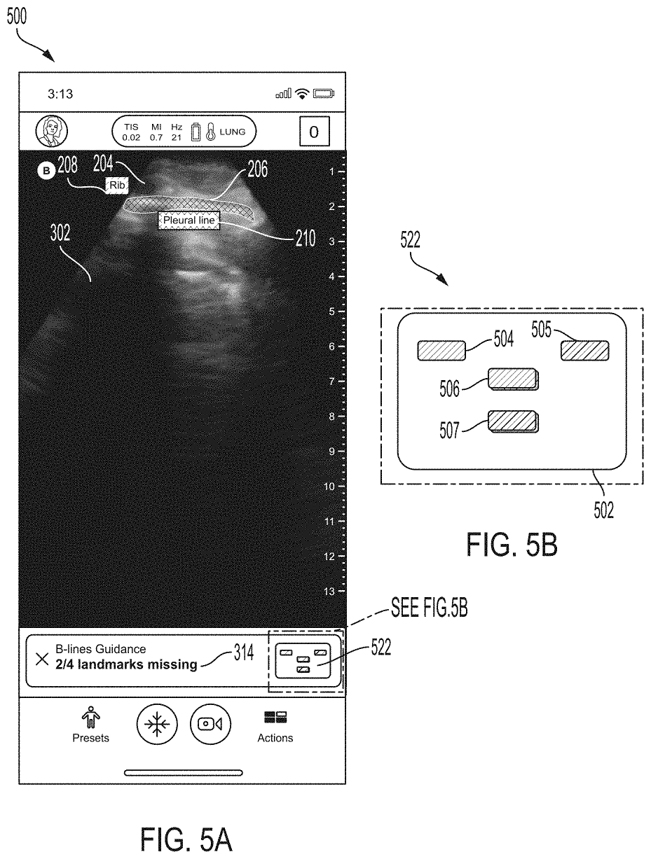

FIG. 5 illustrates a GUI 500. The GUI 500 is the same as the GUI 300, except that the GUI 500 includes the landmark indicator 522 instead of the quality indicator 212. FIG. 5 further includes an inset illustrating the landmark indicator 522 in additional detail. The landmark indicator 522 may be a schematic representation of an ultrasound image of the patient's lungs that includes all the landmarks. The landmark indicator 522 as illustrated in FIG. 5 includes a schematic representation of an ultrasound image of the lungs 502 and schematic representations of landmarks 504-507, in particular schematic representations of a rib 504, another rib 505, the pleural line 506, and A-lines 507. The arrangement of the schematic representations of landmarks 504-507 relative to the schematic representation of the ultrasound image of the lungs 502 and relative to each other may match, at least approximately, the arrangement of the corresponding landmarks relative to an actual ultrasound image of the lungs and relative to each other.

In some embodiments, one or more of the schematic representations of the landmarks 504-507 may be highlighted. For example, in FIG. 5, the processing device displays the schematic representations of the landmarks 505 and 506 in a different color than the schematic representations of the landmarks 504 and 507. Highlighting the segmented landmarks and labels corresponding to the rib and A lines may indicate that these landmarks are missing in the ultrasound image 302. Generally, the processing device may highlight in the landmark indicator 522 those schematic representations of landmarks corresponding to landmarks that are missing in the collected ultrasound image. Referring to the landmark indicator 522 and its highlighted portions may help the user collect a clinically usable ultrasound image.

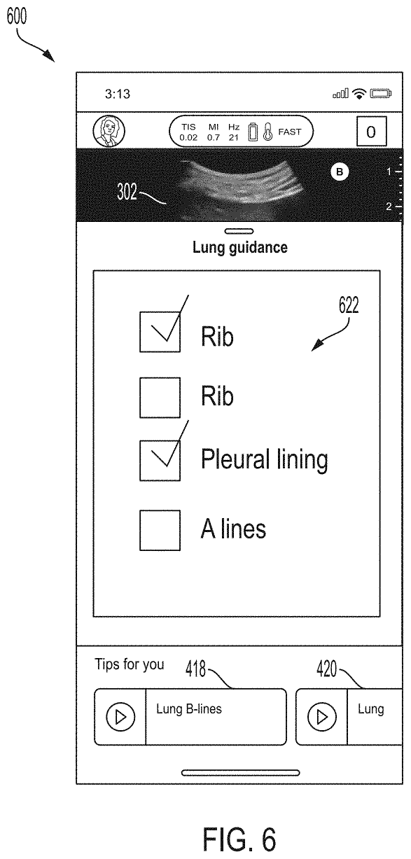

FIG. 6 illustrates a GUI 600. The GUI 600 is the same as the GUI 400, except that the GUI 600 includes a checklist 622 instead of the reference ultrasound image 402. The checklist 622 indicates which of the landmarks are present in the ultrasound image 302 and which are missing.

It should be appreciated that the GUIs 200, 300, 400, 500, and 600 may be specific to lung ultrasound imaging (though embodiments of the present applications are not limited to lung imaging). In particular, the segmented landmarks 204-207, the labels 208-211, any statistical models generating the segmented landmark 204-207 and the labels 208-211, any statistical models computing a quality of the ultrasound images for display by the quality indicators 212 and 214, the reference ultrasound image 402, the landmark indicator 522, and the checklist 622 may be specific to lung imaging. In some embodiments, prior to displaying any of the GUIs described above, a user may select an option associated with lung imaging from a different GUI displayed by the processing device, and the processing device may then use those lung-specific items described above. If a user selected an associated with imaging a different portion of the body, then the processing device may then use items specific to imaging that portion of the body.

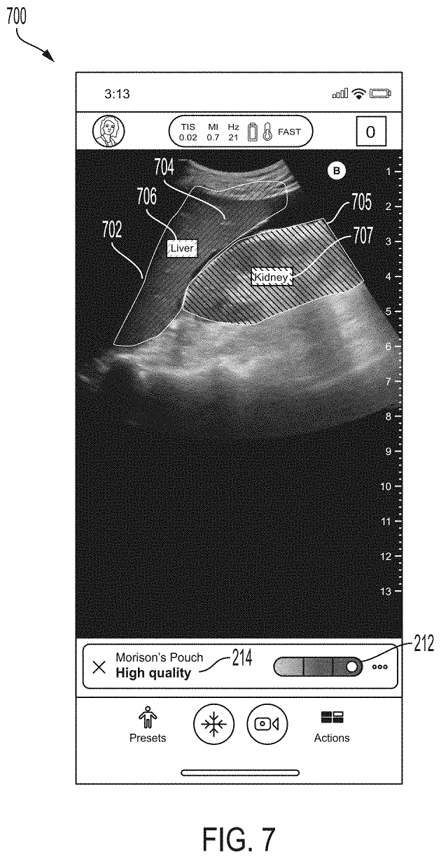

Thus, as described above, some embodiments include GUIs for ultrasound imaging of other portions of the body besides the lungs. FIG. 7 illustrates an example GUI 700. The GUI 700 is the same as the GUI 200, except that the GUI 700 includes an ultrasound image 702, segmented landmarks 704 and 705, and labels 706 and 707. The ultrasound image 402 is an ultrasound image of Morisson's pouch. Further description of ultrasound images may be found with reference to the ultrasound image 202. As indicated by the label 706, the segmented landmark 704 is superimposed on the portion of the ultrasound image 702 depicting the liver. As indicated by the label 707, the segmented landmark 705 is superimposed on the portion of the ultrasound image 702 depicting the kidney. Further description of segmented landmarks may be found with reference to the segmented landmarks 204-2007 and further description of labels may be found with references to the labels 208-211.

As described above, certain ultrasound images may be clinically usable when they include certain landmarks, such as anatomical regions or structures. For example, an ultrasound image of Morisson's pouch, such as the ultrasound image 702, may be clinically usable when it includes the liver and at least one kidney. Similarly, an ultrasound image of a right upper quadrant (RUQ) may be deemed clinically usable when the ultrasound image includes the liver and at least one kidney. As yet another example, an ultrasound image showing a cardiac view (e.g., an apical 4 chamber view) may be deemed clinically usable when the ultrasound image includes the right ventricle (RV), the right atrium (RA), the left ventricle (LV), the left atrium (LA), the mitral valve (MV) and a tricuspid valve (TV). As yet another example, an ultrasound image showing another cardiac view (e.g., a parasternal long axis view (PLAX)) may be deemed clinically usable when the ultrasound image includes the left atrium, the left ventricle, the aortic valve, the mitral valve, the right ventricular outflow tract (RVOT), the descending thoracic aorta (DTA) and the aortic root (AR). As yet another example, an ultrasound image showing yet another cardiac view (e.g., a parasternal short axis view (PSAX)) may be deemed clinically usable when the ultrasound image includes the right ventricle, the left ventricle, one or more papillary muscles, the mitral valve and the aortic valve. As yet another example, an ultrasound image showing yet another cardiac view (e.g., a subxiphoid view) may be deemed clinically usable when the ultrasound image includes the right ventricle, the left ventricle, the right atrium, the left atrium and the liver. As yet another example, an ultrasound image showing a splenorenal recess may be deemed clinically usable when the ultrasound image includes the spleen and at least one kidney. Similarly, an ultrasound image of a left upper quadrant (LUQ) may be deemed clinically usable when the ultrasound image includes the spleen and at least one kidney. As yet another example, an ultrasound image showing a pelvic view may be deemed clinically usable when the ultrasound image includes the bladder. Similarly, an ultrasound image of a bladder may be deemed clinically usable when the ultrasound image includes the entire bladder or at least a fraction of the bladder. As yet another example, an ultrasound image of the carotid artery may be deemed clinically usable when the ultrasound image includes the common carotid artery and the carotid bulb. As yet another example, an ultrasound image of the femoral artery may be deemed clinically usable when the ultrasound image includes the common femoral artery and the femoral bulb.

The presence or absence of such landmarks may correlate with the quality level of the ultrasound image determined by the statistical model described above. For example, the presence of liver and at least one kidney in the ultrasound image 702 may correlate with the high quality determined by the statistical model and displayed by the quality indicators 212 and 214.

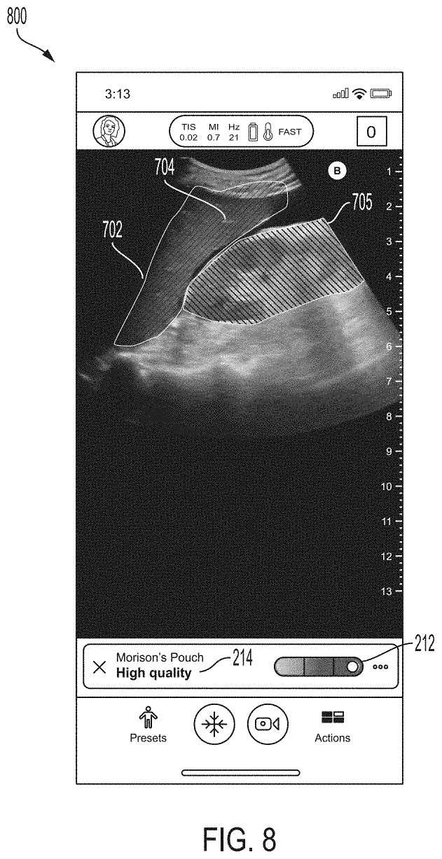

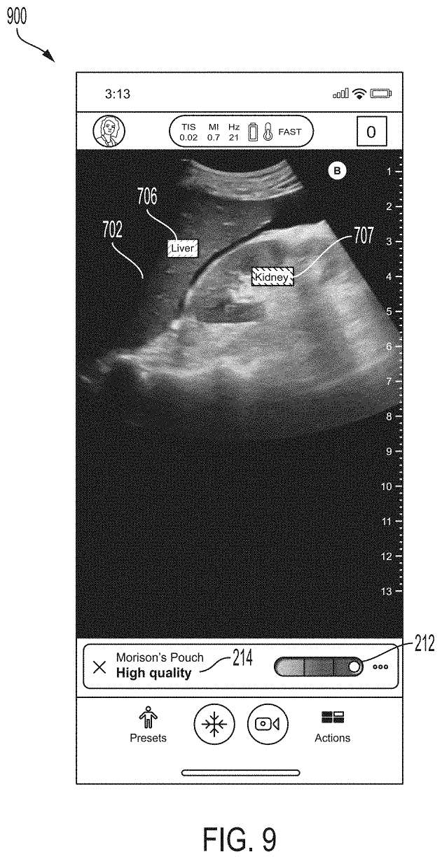

FIGS. 8 and 9 illustrate alternative forms for the GUI 700. It should be appreciated that the alternative forms may also be used for the GUIs 200, 300, 400, 5000, 600, and GUIs for images of any other body portion. FIG. 8 illustrates an example GUI 800. The GUI 800 is the same as the GUI 700, except that the GUI 800 lacks the labels 706 and 707. FIG. 9 illustrates an example GUI 900. The GUI 900 is the same as the GUI 700, except that the GUI 900 lacks the segmented landmarks 704 and 705.

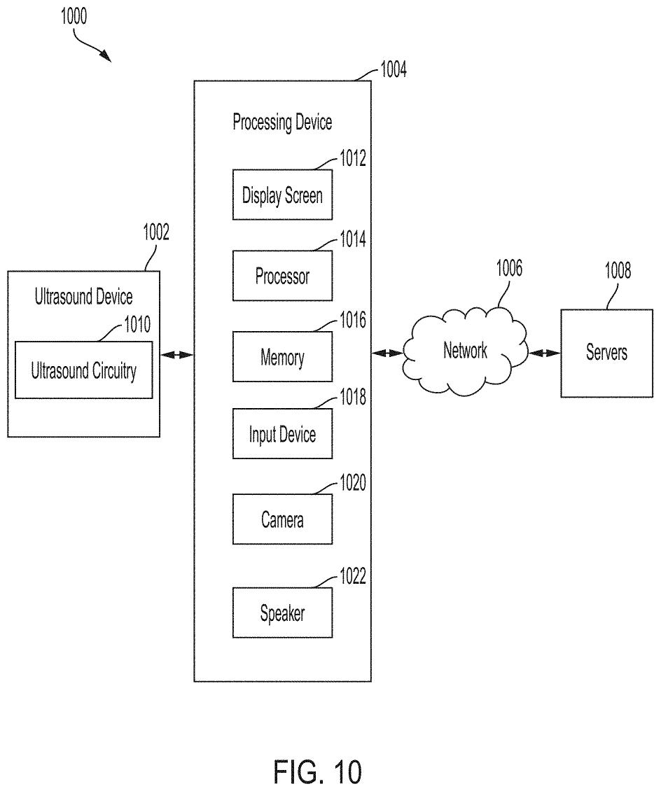

FIG. 10 illustrates a schematic block diagram of an example ultrasound system 1000 upon which various aspects of the technology described herein may be practiced. The ultrasound system 1000 includes an ultrasound device 1002, a processing device 1004, a network 1006, and one or more servers 1008. The processing device 1004 may be any of the processing devices described herein. The ultrasound device 1002 may be any of the ultrasound devices described herein.

The ultrasound device 1002 includes ultrasound circuitry 1010. The processing device 1004 includes a camera 1020, a display screen 1012, a processor 1014, a memory 1016, an input device 1018, and a speaker 1022. The processing device 1004 is in wired (e.g., through a lightning connector or a mini-USB connector) and/or wireless communication (e.g., using BLUETOOTH, ZIGBEE, and/or WiFi wireless protocols) with the ultrasound device 1002. The processing device 1004 is in wireless communication with the one or more servers 1008 over the network 1006.

The ultrasound device 1002 may be configured to generate ultrasound data that may be employed to generate an ultrasound image. The ultrasound device 1002 may be constructed in any of a variety of ways. In some embodiments, the ultrasound device 1002 includes a transmitter that transmits a signal to a transmit beamformer which in turn drives transducer elements within a transducer array to emit pulsed ultrasonic signals into a structure, such as a patient. The pulsed ultrasonic signals may be back-scattered from structures in the body, such as blood cells or muscular tissue, to produce echoes that return to the transducer elements. These echoes may then be converted into electrical signals by the transducer elements and the electrical signals are received by a receiver. The electrical signals representing the received echoes are sent to a receive beamformer that outputs ultrasound data. The ultrasound circuitry 1010 may be configured to generate the ultrasound data. The ultrasound circuitry 1010 may include one or more ultrasonic transducers monolithically integrated onto a single semiconductor die. The ultrasonic transducers may include, for example, one or more capacitive micromachined ultrasonic transducers (CMUTs), one or more CMOS (complementary metal-oxide-semiconductor) ultrasonic transducers (CUTs), one or more piezoelectric micromachined ultrasonic transducers (PMUTs), and/or one or more other suitable ultrasonic transducer cells. In some embodiments, the ultrasonic transducers may be formed on the same chip as other electronic components in the ultrasound circuitry 1010 (e.g., transmit circuitry, receive circuitry, control circuitry, power management circuitry, and processing circuitry) to form a monolithic ultrasound device. The ultrasound device 1002 may transmit ultrasound data and/or ultrasound images to the processing device 1004 over a wired (e.g., through a lightning connector or a mini-USB connector) and/or wireless (e.g., using BLUETOOTH, ZIGBEE, and/or WiFi wireless protocols) communication link.

Referring now to the processing device 1004, the processor 1014 may include specially-programmed and/or special-purpose hardware such as an application-specific integrated circuit (ASIC). For example, the processor 1014 may include one or more graphics processing units (GPUs) and/or one or more tensor processing units (TPUs). TPUs may be ASICs specifically designed for machine learning (e.g., deep learning). The TPUs may be employed, for example, to accelerate the inference phase of a neural network. The processing device 1004 may be configured to process the ultrasound data received from the ultrasound device 1002 to generate ultrasound images for display on the display screen 1012. The processing may be performed by, for example, the processor 1014. The processor 1014 may also be adapted to control the acquisition of ultrasound data with the ultrasound device 1002. The ultrasound data may be processed in real-time during a scanning session as the echo signals are received. In some embodiments, the displayed ultrasound image may be updated a rate of at least 5 Hz, at least 10 Hz, at least 20 Hz, at a rate between 5 and 60 Hz, at a rate of more than 20 Hz. For example, ultrasound data may be acquired even as images are being generated based on previously acquired data and while a live ultrasound image is being displayed. As additional ultrasound data is acquired, additional frames or images generated from more-recently acquired ultrasound data may be sequentially displayed. Additionally, or alternatively, the ultrasound data may be stored temporarily in a buffer during a scanning session and processed in less than real-time.

The processing device 1004 may be configured to perform certain of the processes (e.g., the process 100) described herein using the processor 1014 (e.g., one or more computer hardware processors) and one or more articles of manufacture that include non-transitory computer-readable storage media such as the memory 1016. The processor 1014 may control writing data to and reading data from the memory 1016 in any suitable manner. To perform certain of the processes described herein, the processor 1014 may execute one or more processor-executable instructions stored in one or more non-transitory computer-readable storage media (e.g., the memory 1016), which may serve as non-transitory computer-readable storage media storing processor-executable instructions for execution by the processor 1014. The camera 1020 may be configured to detect light (e.g., visible light) to form an image. The camera 1020 may be on the same face of the processing device 1004 as the display screen 1012. The display screen 1012 may be configured to display images and/or videos, and may be, for example, a liquid crystal display (LCD), a plasma display, and/or an organic light emitting diode (OLED) display on the processing device 1004. The input device 1018 may include one or more devices capable of receiving input from a user and transmitting the input to the processor 1014. For example, the input device 1018 may include a keyboard, a mouse, a microphone, touch-enabled sensors on the display screen 1012, and/or a microphone. The display screen 1012, the input device 1018, the camera 1020, and the speaker 1022 may be communicatively coupled to the processor 1014 and/or under the control of the processor 1014.

It should be appreciated that the processing device 1004 may be implemented in any of a variety of ways. For example, the processing device 1004 may be implemented as a handheld device such as a mobile smartphone or a tablet. Thereby, a user of the ultrasound device 1002 may be able to operate the ultrasound device 1002 with one hand and hold the processing device 1004 with another hand. In other examples, the processing device 1004 may be implemented as a portable device that is not a handheld device, such as a laptop. In yet other examples, the processing device 1004 may be implemented as a stationary device such as a desktop computer. The processing device 1004 may be connected to the network 1006 over a wired connection (e.g., via an Ethernet cable) and/or a wireless connection (e.g., over a WiFi network). The processing device 1004 may thereby communicate with (e.g., transmit data to or receive data from) the one or more servers 1008 over the network 1006. For example, a party may provide from the server 1008 to the processing device 1004 processor-executable instructions for storing in one or more non-transitory computer-readable storage media (e.g., the memory 1016) which, when executed, may cause the processing device 1004 to perform certain of the processes (e.g., the process 100) described herein.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified.

Use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

As used herein, reference to a numerical value being between two endpoints should be understood to encompass the situation in which the numerical value can assume either of the endpoints. For example, stating that a characteristic has a value between A and B, or between approximately A and B, should be understood to mean that the indicated range is inclusive of the endpoints A and B unless otherwise noted.

The terms “approximately” and “about” may be used to mean within ±20% of a target value in some embodiments, within ±10% of a target value in some embodiments, within ±5% of a target value in some embodiments, and yet within ±2% of a target value in some embodiments. The terms “approximately” and “about” may include the target value.

Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

Having described above several aspects of at least one embodiment, it is to be appreciated various alterations, modifications, and improvements will readily occur to those skilled in the art. Such alterations, modifications, and improvements are intended to be object of this disclosure. Accordingly, the foregoing description and drawings are by way of example only.

What is claimed is:

1. An apparatus, comprising:
a processing device configured to:
generate an ultrasound image for display;
determine a quality of the ultrasound image;
determine whether the quality of the ultrasound image is below a threshold quality;
if the processing device determines that the quality of the ultrasound image is not below the threshold quality, provide an indication of the quality of the ultrasound image; and
if the processing device determines that the quality of the ultrasound image is below the threshold quality:
determine one or more missing landmarks in the ultrasound image; and
provide an indication of the quality of the ultrasound image and an indication of the one or more missing landmarks in the ultrasound image.

2. The apparatus of claim 1, wherein the one or more missing landmarks comprise one or more anatomical features that, if present in the ultrasound image, would make the ultrasound image clinically usable.

3. The apparatus of claim 1, wherein the ultrasound image comprises an ultrasound image of lungs, and the one or more missing landmarks comprise one or more of ribs, a pleural line, and A lines.

4. The apparatus of claim 1, wherein the ultrasound image comprises an ultrasound image of Morison's pouch, and the one or more missing landmarks comprise on or more of a liver and a kidney.

5. The apparatus of claim 1, wherein the ultrasound image comprises an ultrasound image showing an apical 4 chamber view, and the one or more missing landmarks comprises one or more of a right ventricle, a right atrium, a left ventricle, a left atrium, a mitral valve and a tricuspid valve.

6. The apparatus of claim 1, wherein the ultrasound image comprises an ultrasound image showing a parasternal long axis view (PLAX), and the one or more missing landmarks comprises one or more of a left atrium, a left ventricle, an aortic valve, a mitral valve, a right ventricular outflow tract, a descending thoracic aorta and am aortic root.

7. The apparatus of claim 1, wherein the ultrasound image comprises an ultrasound image showing a parasternal short axis view (PSAX), and the one or more missing landmarks comprises one or more of a right ventricle, a left ventricle, a papillary muscle, a mitral valve and an aortic valve.

8. The apparatus of claim 1, wherein the ultrasound image comprises an ultrasound image showing a subxiphoid view, and the one or more missing landmarks comprises one or more of a right ventricle, a left ventricle, a right atrium, a left atrium a liver.

9. The apparatus of claim 1, wherein the ultrasound image comprises an ultrasound image of a splenorenal recess, and the one or more missing landmarks comprises one or more of a spleen and a kidney.

10. The apparatus of claim 1, wherein the ultrasound image comprises an ultrasound image showing a pelvic view, and the one or more missing landmarks comprises a bladder.

11. The apparatus of claim 1, wherein the ultrasound image comprises an ultrasound image of a carotid artery, and the one or more missing landmarks comprises a common carotid artery and a carotid bulb.

12. The apparatus of claim 1, wherein the ultrasound image comprises an ultrasound image of a femoral artery, and the one or more missing landmarks comprises a common femoral artery and a femoral bulb.

13. The apparatus of claim 1, wherein the processing device is configured, when providing the indication of the one or more missing landmarks in the ultrasound image, to display a number of the missing landmarks.

14. The apparatus of claim 1, wherein the processing device is configured, when providing the indication of the one or more missing landmarks in the ultrasound image, to display:
a number of total landmarks that, if present in the ultrasound image, would make the ultrasound image clinically usable, and
a number of landmarks present in the ultrasound image.

15. The apparatus of claim 1, wherein the processing device is configured, when providing the indication of the one or more missing landmarks in the ultrasound image, to display a schematic representation of an ultrasound image that includes schematic representations of all landmarks that, if present in the ultrasound image, would make the ultrasound image clinically usable.

16. The apparatus of claim 1, wherein the processing device is configured, when determining the quality of the ultrasound image, to determine the quality based on a predicted proportion of experts who would consider the ultrasound image to be clinically usable.

17. A method, comprising:
generating an ultrasound image for display;
determining a quality of the ultrasound image;
determining whether the quality of the ultrasound image is below a threshold quality;
if it is determined that the quality of the ultrasound image is not below the threshold quality, providing an indication of the quality of the ultrasound image; and
if it is determined that the quality of the ultrasound image is below the threshold quality:
determining one or more missing landmarks in the ultrasound image; and
providing an indication of the quality of the ultrasound image and an indication of the one or more missing landmarks in the ultrasound image.

18. An apparatus comprising:
a processing device configured to:
obtain data representing an ultrasound image; and
determine whether the ultrasound image is clinically usable, wherein the determining comprises determining whether the ultrasound image lacks one or more landmarks.

19. The apparatus of claim 18, wherein determining whether the ultrasound image is clinically usable further comprises determining a quality value representative of a quality of the ultrasound image and comparing the quality value to a threshold quality value.

20. The apparatus of claim 18, wherein the one or more landmarks comprise one or more anatomical features.
